(19) United States
(12) Patent Application Publication
(10) Pub. No.: US 2006/0129432 A1
(43) Pub. Date: Jun. 15, 2006

(54) Device, system, and method for providing health management service

(75) Inventors: Yoon-sun Choi, Seoul (KR); Young-soo Kim, Seoul (KR); Jung-min Yoon, Seoul (KR); Sung-won Ahn, Seoul (KR); Do-gyoon Kim, Seongnam-si (KR); Sang-cheol Moon, Seoul (KR); Ho-seung Lee, Seongnam-si (KR)

Correspondence Address: SUGHRUE MION, PLLC, 2100 PENNSYLVANIA AVENUE, N.W., SUITE 800, WASHINGTON, DC, 20037, US

(73) Assignee: SAMSUNG ELECTRONICS CO., LTD.

(21) Appl. No.: 11/296,338

(22) Filed: Dec. 8, 2005

(30) Foreign Application Priority Data
Dec. 9, 2004 (KR) 10-2004-0103564

(51) Int. Cl. A61B 5/00

(52) U.S. Cl. 705/3; 600/300

(57) ABSTRACT

A device, system, and method for providing health management services are provided. The device, system, and method analyze health data based on user health data obtained through a health measurement device and provide feedback data based on the analyzed result, thereby allowing accurate monitoring and management of the user's health status.

### CROSS-REFERENCE TO RELATED APPLICATION

This application is based upon and claims the benefit of priority from Korean Patent Application No. 10-2004-0103564 filed on Dec. 9, 2004 in the Korean Intellectual Property Office, the disclosure of which is incorporated herein by reference in its entirety.

### BACKGROUND OF THE INVENTION

1. Field of the Invention

Devices, systems, and methods consistent with the present invention related to providing health management services, and more particularly, to providing health management services including analyzing data based on user health data obtained by a health measurement apparatus, providing feedback data according to the result of analysis, monitoring user's compliance with the feedback data, and applying a weighted value according to the result of monitoring for re-prescription.

2. Description of the Related Art

Recent years have witnessed the emergence and rapid proliferation of wireless communication environments (e.g., Ipv6, wireless local area network (WLAN), Bluetooth, etc.), sensing and home networking technology based on these wireless communication environments, and various technology standardizations. The technology standardizations include, for example, the Health Level Seven (HL7), which is an encoding method for patient medical records which include billings data and chart data, the Health Insurance Portability and Accountability Act (HIPPA), the IEEE 1073 standards organization, which is organized to define an IEEE standard for networking and protocols for collecting and transmitting clinical observation data, and so on. Additionally, technological advancement in data analysis through real time data collection and data analysis tools has enabled precise, convenient monitoring of health state and personalized health management services.

To meet more sophisticated needs and expectations of customers arising from these consumers' growing interests in and awareness of health care services, a health care provider tends to focus on convenience and customization in use of the health care services and systems associated therewith. In this regard, the desire for personalized health management business providing services, such as lifestyle disease prevention or weight management based on individual's accumulated health data, has increased.

While related-art health management services focus only on health care activities such as treatment of disease for patients in hospitals or medical institutions, consumer's needs for health management are now changing toward prevention of disease and maintenance of health for a healthy person. That is, as increasing attention is directed toward quality of life and well-being/wellness, preventive health management services such as measurement of health state or management of exercise amount suitable for individual health needs are gaining popularity.

In this regard, there is an urgent need for a personalized total solution for health management that provides integrated products and services through monitoring of an individual's health. However, health management service providers tend to focus on separate, product-oriented services while having given little attention to a personalized total solution, so that they are unable to meet changing technical environment and consumer needs.

FIG. 1 is a schematic diagram of a related-art PC- and Internet-based telemedicine and health management system.

The related-art telemedicine and health management system includes a plurality of health measurement devices 10 measuring an individual's health state, an individual personal computer (PC) terminal 20 that is connected with a wired cable such as Universal Serial Bus or Serial to receive health measurement data and transmit the received data to a service server 30, and the service server 30 collecting the received data and providing appropriate service based on the collected data.

FIG. 2 is an internal block diagram of a related-art PC- and Internet-based telemedicine and health management system.

Referring to FIG. 2, the health measurement devices 10a-10n routinely check a user's health state and transmit the user's health state to the PC 20 on a wired cable for recording. To accomplish these functions, each health measurement device 10a includes a device-embedded application 13a, a device connection module 11a, and an USB/Serial port 12a.

The PC 20 acts as a gateway to collect user information for each of health measurement devices 10a through 10n, and to transmit the collected user information to a health management service server 30. To accomplish this function, the PC 20 includes: (1) a device connection module 21 for receiving data from the health measurement devices 10a-10n; (2) PC applications 24 and 25, respectively for device 10a and device 10n, for storing and processing the received data; and (3) a local area network (LAN) 23 acting as an interface for transmitting the data received from the health measurement devices 10a-10n to the health management service server 30.

The health management service server 30 provides a wide variety of services based on the data received from the health measurement devices 10a-10n and includes a LAN 31 acting as an interface for receiving data from the PC 20 and a service application 32 analyzing, processing, and storing the received data.

However, the related-art method for providing PC- and Internet-based telemedicine and health management services through the system having the above-mentioned configuration has a problem in that each device must be purchased from a different manufacturer when a plurality of devices are used.

Further, the related-art method involves installing a necessary program to a device, connecting the device to a PC for transmission of data obtained by the device to a service server, and independently receiving feedback data for each device, thus resulting in user inconvenience.

Another drawback of the related-art method is that an integrated service or integrated management for health measurement devices is impossible or difficult to perform because the approach makes it impossible to exchange data measured by different health measurement devices.

Further, services provided by the related-art method are limited to transmitting health data simply obtained through measurement, providing necessary information and services through restricted analysis, and performing intermittent monitoring of user's health state upon request from a user. Thus, the related-art method cannot provide a personalized total solution.

In addition, the related-art method tends to provide transient services as needed rather than ongoing measurement and management of personal health states.

In addition to the above described related-art method, the operation of a related-art network-based health management system will now be described.

First, unspecified customers access the website of a health enhancement system via a network and register as members by entering personal information in a predetermined member registration form.

Then, a member card or a fingerprint recognition system located at a predetermined place is used to perform member authentication for health examination, an external institution is commissioned to perform an overall health examination, and the result of the overall examination is stored in a database (DB).

Next, an appropriate service is provided to a user based on the result of analyzing the examination result and the user subsequently checks the analyzed examination result to verify his/her health state.

However, a related-art network-based health management approach requires a user to visit a medical/clinical institution to measure health data, thus making it difficult to take timely and appropriate action in response to real time change in health data.

Further, the related-art system has a problem in that it is not satisfactory to monitor user compliance with a prescription, and to provide customized services that can be tailored to the individual's needs for re-prescription based on the result of monitoring.

In other words, the related-art approach tends to provide transient services as needed rather than ongoing measurement and management of health state by simply converting off-line treatment and health management services into on-line services.

In another related-art system, Korean Patent Application No. 10-2000-052391 proposes another network-based health management system. This proposed health management system is configured to provide exercise and dietary prescription based on individual's health management data that is made available through other medical institutions or medical service providers. However, this health management system has a problem in that monitoring of compliance with prescription and providing re-prescription customized services to satisfy the individual needs are not satisfactory.

### BRIEF SUMMARY OF THE INVENTION

The invention provides a method for analyzing user health data obtained through a health measurement device and providing feedback based on the analyzed result.

The invention also provides a method for providing health management service and health prescription suitable for a user by monitoring user health data and feedback data and assigning a weighted value according to the result of monitoring for re-prescription.

The invention also provides a method for measuring user health data and providing health planner's one-to-one guidance based on the health data.

The above stated objects as well as other objects, features and advantages, of the invention will become clear to those skilled in the art upon review of the following description.

According to an aspect of the invention, there is provided a health measurement device including a measurement module, a user authentication module, a user management module, and a protocol module.

According to an aspect of the invention, there is provided a health management station including a measurement module measuring user's health status, a user management module managing information concerning a user who uses a health measurement device based on the information, a data management module managing user health data obtained through the measurement and feedback data received from a health management service server, and a protocol module transmitting the health data and the feedback data.

According to another aspect of the invention, there is provided a health management service server including a user management module managing user information received from a health management station, a data analysis module analyzing user's health status based on the user information and health data received from the health management station and generating feedback data based on the analyzed result, a data management module managing the user health data received from the health management station and the feedback data, and a protocol module transmitting the health data and the feedback data.

According to still another aspect of the invention, there is provided a health guide station including a device connection module transmitting and receiving guide information received from a health planner and user health data, a user authentication module reading user identification (ID) information and authenticating the user, a measurement module measuring user's health status, a data management module managing health data obtained through the measurement, the guide information, and health data and feedback data received from a health management service server, a protocol module transmitting the guide information, the health data, and the user information.

According to yet another aspect of the invention, there is provided a system for providing a health management service, including a health measurement device measuring user's health status and transmitting health data obtained through the measurement to a health management station; a health management station transmitting the user health data received from the health measurement device to a health management service server and managing feedback data received from the health management service server, a health management service server analyzing the user health data received from the health management station and managing the analyzed result and feedback data generated based on the analyzed result, and a health guide station measuring user health data and transmitting guide information received from a health planner to the health management service server.

According to a further aspect of the invention, there is provided a system for providing a health management service including a health management service server analyzing user health data and managing the analyzed result and feedback data generated based on the analyzed result, and an external service server receiving user information, health data, and feedback data from the health management service server and providing service appropriate for the user based on the received user information, health data, and feedback data.

According to yet a further aspect of the invention, there is provided a method for providing a health management service, including measuring user's health status through a health measurement device, the health measurement device reading user ID information and authenticating the user, transmitting health data obtained through the measurement and user information about the authenticated user to a health management station, storing the user health data and user information and transmitting the user health data and user information to a health management service server, analyzing the received user health data and generating feedback data based on the analyzed result, and transmitting the feedback data to the health management station.

According to still yet another aspect of the invention, there is provided a method for providing a health management service, including receiving user information, health data, and feedback data from a health management service server, searching for information concerning a service suitable for the user based on the received user information, health data, and feedback data; and providing the found service information to the user.

### BRIEF DESCRIPTION OF THE DRAWINGS

The above and other objects, features and advantages of the invention will become more apparent by describing in detail exemplary embodiments thereof with reference to the attached drawings in which:

FIG. 1 is a schematic diagram of a related-art PC- and Internet-based telemedicine and health management system;

FIG. 2 is an internal block diagram of a related-art PC- and Internet-based telemedicine and health management system;

FIG. 3 shows a system for providing health management service according to a first exemplary embodiment of the invention;

FIG. 4 is an internal block diagram of the health measurement device in the system for providing health management service of FIG. 3;

FIG. 5 is an internal block diagram of the health management station in the system for providing health management service of FIG. 3;

FIG. 6 is an internal block diagram of the health management service server in the system for providing health management service of FIG. 3;

FIG. 7 is an internal block diagram of the health guide station in the system for providing health management service of FIG. 3;

FIG. 8 is an internal block diagram of the authenticating device in the system for providing health management service of FIG. 3;

FIG. 9 is a diagram showing a system for providing a health management service consisting of a health management service server and external service servers according to a second exemplary embodiment of the invention;

FIG. 10 shows a health management screen displayed on a health management station according to an exemplary embodiment of the invention;

FIGS. 11A, 11B and 11C illustrate a process of providing information about food items in a user's meal using a mobile measurement device according to an exemplary embodiment of the invention;

FIG. 12 is a flowchart illustrating a method for providing health management service according to an exemplary embodiment of the invention;

FIG. 13 is a flowchart illustrating the process of providing health management service through a health guide station in the method for providing health management service according to an exemplary embodiment of the invention; and

FIG. 14 is a flowchart illustrating the process of providing re-prescription service to the user in the method for providing health management service according to an exemplary embodiment of the invention.

### DETAILED DESCRIPTION OF EXEMPLARY EMBODIMENTS OF THE INVENTION

The invention may be understood more readily by reference to the following detailed description of exemplary embodiments and the accompanying drawings. The described exemplary embodiments are intended to assist the understanding of the invention. The invention may, however, be embodied in many different forms and should not be construed as being limited to the exemplary embodiments set forth herein. Rather, these exemplary embodiments are provided so that this disclosure will be thorough and complete and will fully convey the concept of the invention to those skilled in the art, and the invention will only be defined by the appended claims. Like reference numerals refer to like elements throughout the specification.

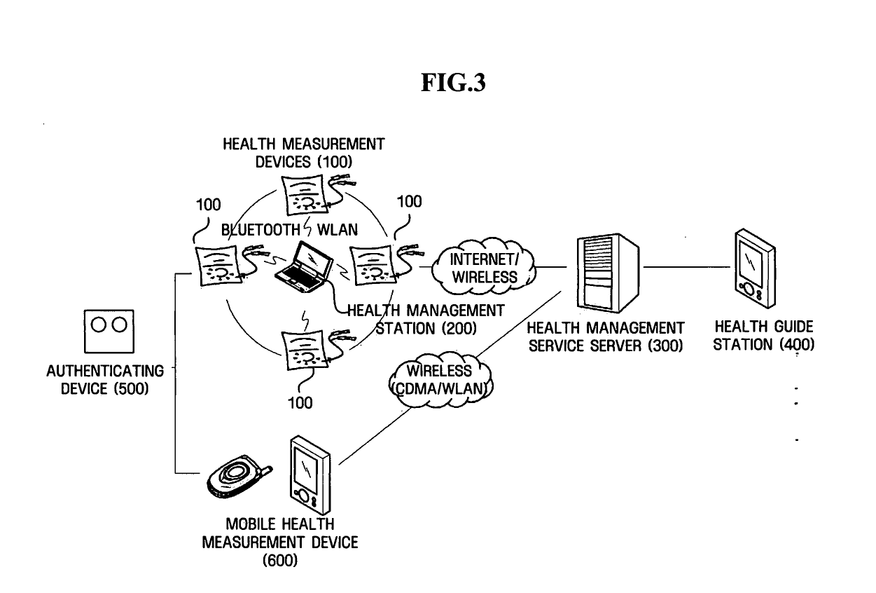

FIG. 3 shows a system for providing health management service according to a first exemplary embodiment of the invention.

Referring to FIG. 3, a system for providing a health management service includes at least one health measurement device 100, a health management station 200, a health management service server 300, a health guide station 400, and an authenticating unit 500. The system further includes a mobile health measurement device 600.

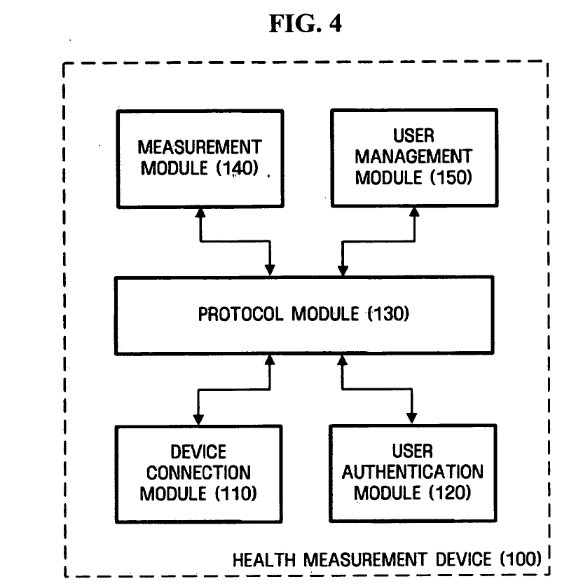

The health measurement device 100 measures a user's health state and transmits health data obtained through the health measurement device 100 to a health management station 200 via wireless communications such as Bluetooth or wireless local area network (WLAN). Of course, the health data may be transmitted via wired communications. The configuration and operation of the health measurement device 100 will be described in more detail later with reference to FIG. 4.

The health measurement device 100 can be understood as a blood pressure measurement device, a blood sugar measurement device, a heartbeat measurement device, a weight scale, a body fat measurement device, a calorie tracker, or an exercise machine such as a treadmill, a stepper, and a cycle.

The health management station 200 manages the user health data received from the health measurement device 100 and transmits the health data to the health management service server 300.

The health management station 200 also receives feedback data obtained through analysis of the health data from the health management service server 300 and manages the received feedback data. Here, the health data and the feedback data are transmitted and received via wired communications such as wired cable (e.g., Universal Serial Bus (USB) and Serial), a LAN or wireless communications.

The health management station 200 includes an embedded health management device to measure a user's health state. Here, the embedded health measurement device can be understood as a blood pressure measurement device or a pulsimeter.

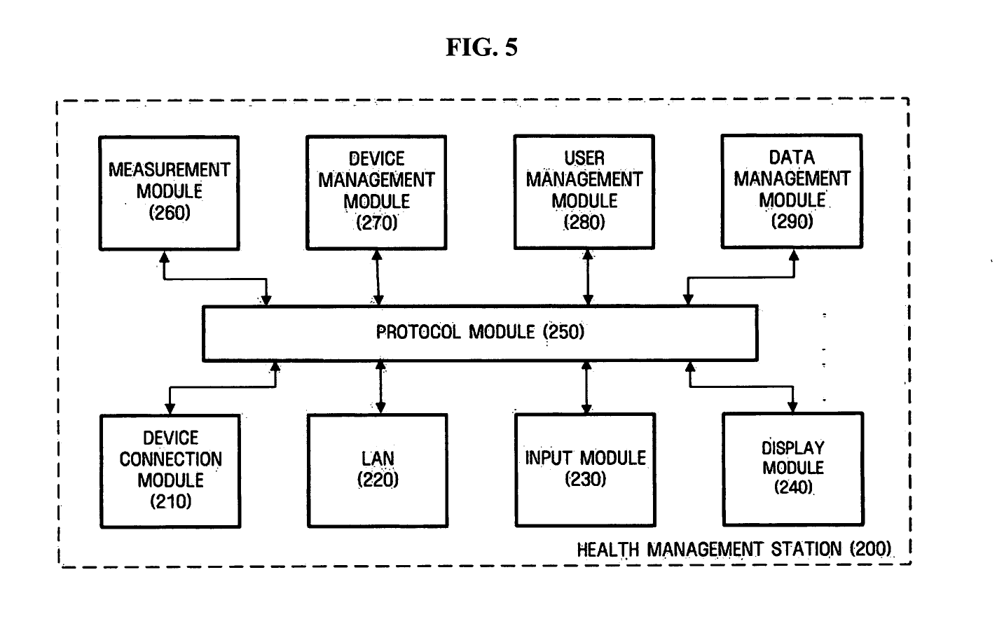

The health management station 200 also displays the received feedback data on its screen or transmits the feedback data to a predetermined health measurement device 100. The configuration and operation of the health management station 200 will be described in more detail later with reference to FIG. 5.

The health management service server 300 analyzes the user health data received from the health management station 200, generates feedback data as the result of the analysis, and transmits the generated feedback data to the health management station 200 and the mobile health measurement device 600.

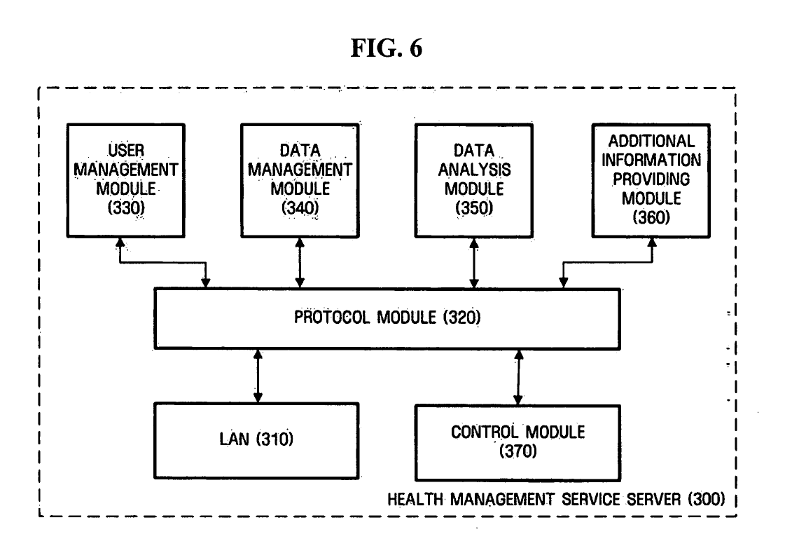

Upon request from the health guide station 400, the health management service server 300 sends the user health data and feedback data to the health guide station 400 while receiving guide information from the health guide station 400 and storing the received guide information. The configuration and operation of the health management service server 300 will be described in more detail below with reference to FIG. 6.

The health guide station 400 receives guide information from a health planner and transmits the received guide information to the health management service server 300. Here, the guide information can be understood as user health data and prescription information containing exercise and dietary recommendations made by the health planner based on the user health data.

The health guide station 400 also performs a predetermined measurement function to measure the user's health status.

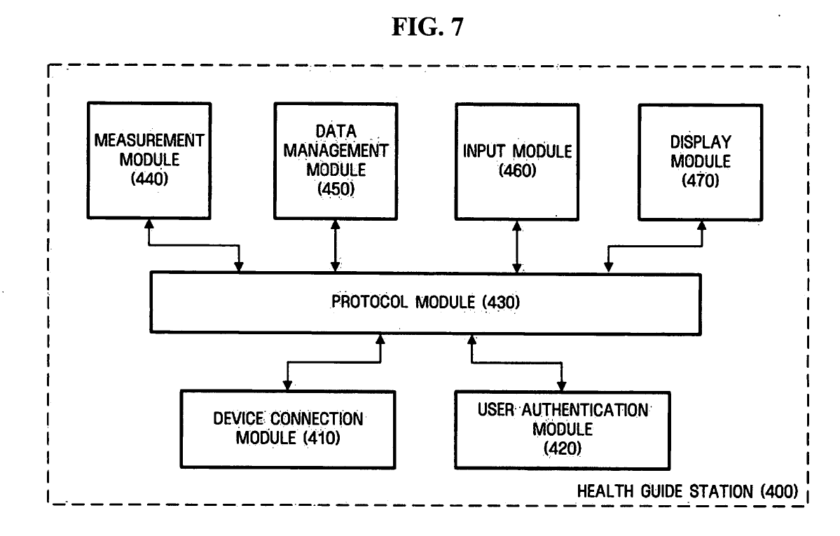

The health guide station 400 requests the health management service server 300 to transmit the user health data and the feedback data and receives the requested data that are then used for creating the guide information. The configuration and operation of the health guide station 400 will be described in more detail below with reference to FIG. 7.

Meanwhile, the health guide station 400 operates through the health planner. The health planner checks a user's health status based on the user health data and provides appropriate guide information containing exercise and dietary recommendations made based on the result of this check.

For example, the health planner makes health inquiry and consultations with a user using telephone and Internet while providing common exercise routines, psychological counseling, and massage off line.

Thus, the health planner makes one-to-one consultations with the user on an on/off line, checks and analyzes the health status of the user, and provides real-time guide information.

For example, the health planner may measure user health data while doing exercise with the user and transmit the health data to the health management service server 300 through the health guide station 400.

The authenticating device 500 stores user data (user demographic information and ID information) and health data and transmits the user data to the health guide station 400 via wireless communications.

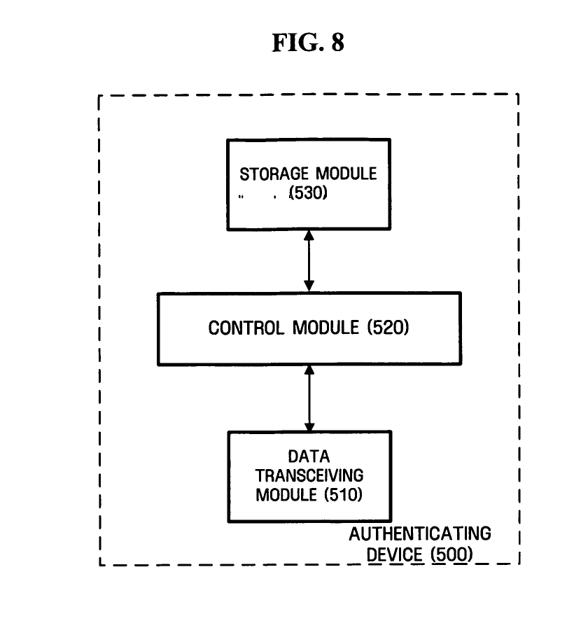

Here, the authenticating device 500 can be understood as a card, a key holder, a bracelet, or other various types of Radio Frequency Identification (RFID) tags containing user personal information. An RFID tag is recognized by the health measurement device 100 and the health guide station 400 on a non-contact basis to authenticate a user. The configuration and operation of the authenticating device 500 will be described in more detail below with reference to FIG. 8.

The mobile health measurement device 600 is used to measure a user's health status and transmit user health data obtained through the measurement to the health management service server 300. Here, the mobile health measurement device 600 can be understood as a mobile phone or PDA. For example, the mobile health measurement device 600 may receive a menu containing food items in a meal that the user has consumed when the user transmits the menu to the health management service server 300, thus enabling the health management service server to monitor user's menu in real time.

The operation of the system for providing a health management service will now be described. A plurality of health measurement devices 100 measure various ones of a user's health states, and transmit health data obtained through the measurements to the health management station 200.

The health management station 200 collects the received health data and transmits the collected health data and user data to the health management service server 300.

Then, the health management service server 300 stores the received health data and user data, analyzes the health data to generate feedback data based on the analyzed result, and transmits the feedback data to the health management station 200. Thus the user is allowed to receive the result of analyzing his/her health state and prescription (feedback data) obtained based on the analyzed result.

FIG. 4 is an internal block diagram of the health measurement device in the system for providing health management service of FIG. 3.

Referring to FIG. 4, the health measurement device 100 includes a device connection module 110, a user authentication module 120, a protocol module 130, a measurement module 140, and a user management module 150.

The device connection module 110 is for connecting the health measurement device 100 with the health management station 200, to transmit the health data to the health management station 200. The device connection module 110 can be a wireless connection module such as Bluetooth or WLAN. However, the health measurement device 100 may also connect with the health management station 200 via a wired link such as wired cable (USB or Serial) or LAN.

The user authentication module 120 reads the user ID information from the authenticating device 500 and authenticates the current user using the health measurement device 100. That is, the user authentication module 120 reads user information and a user authentication key from a RFID tag in the authenticating device 500 in order to authenticate the user.

The protocol module 130 transmits the health data obtained by measuring the user's health state and user information to the device connection module 110. That is, the protocol module 130 converts the user ID information and the user health data obtained through the measurement module 140 into a form that can be available on the health management station 200, and transmits the resulting information to the device connection module 110.

The user measurement module 140 measures the user's health status to generate health data that is then stored in the user management module 150 and the health management station 200.

The user management module 150 stores and maintains information about a user who uses the health measurement device 100 and the user health data obtained through the measurement module 140.

FIG. 5 is an internal block diagram of the health management station in the system for providing the health management service of FIG. 3.

Referring to FIG. 5, the health management station 200 includes a device connection module 210, a LAN 220, an input module 230, a display module 240, a protocol module 250, a measurement module 260, a device management module 270, a user management module 280, and a data management module 290.

The device connection module 210 is for connecting the health measurement device 100 with the health management station 200, to transmit the health data to the health management station 200. The device connection module 210 can be a wireless connection module such as Bluetooth or WLAN. However, the health measurement device 100 may connect with the health management station 200 via a wired link such as wired cable (USB or Serial) or LAN.

The LAN 220 connects the health management station 200 to the health management service server 300 through the Internet. Although FIG. 5 shows a LAN is used as an interface for connecting the health management station 200 with the health management service server 300, a WLAN or wireless application protocol (WAP) conforming to the 802.11 standard may also be used as the interface.

The input module 230 receives predetermined information such as demographic information (e.g., age, gender, etc.) and health status information (e.g., height, weight, blood pressure, etc.) from a user. For example, the user may enter his/her demographic information or health status information such as the amount of caloric intake and target weight for authentication.

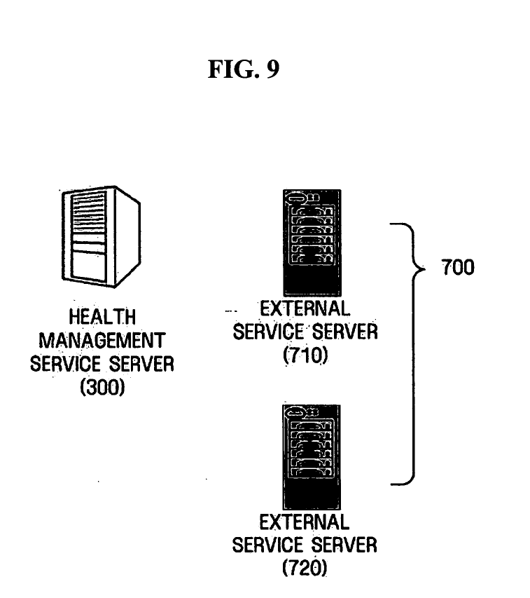

The display module 240 displays a health management screen and feedback data received from the health management service server 300. Here, the health management screen includes multiple menu items, such as “daily health inquiry,” “physical strength measurement,” “prescription,” “diary,” “my health,” “blood pressure measurement,” and “log in/out,” which will be described in detail later with reference to FIG. 9.

The protocol module 250 transmits the health data obtained by measuring the user's health state and user information. That is, the protocol module 250: (1) converts the user information and the user health data received from the device connection module 210 into a form that can be available on the user management module 280 and the data management module 290; (2) converts the user ID information and the user health data obtained through the measurement module 210 into a form that can be available on the health management service server 300; and (3) transmits the resulting information to the device connection module 210.

The measurement module 260 measures the user's health status to generate health data that is then stored in the user management module 150 and the health management station 200. For example, the measurement module 260 measures a user's blood pressure and pulse.

The device management module 270 manages the plurality of health measurement devices 100 by updating embedded applications of the plurality of health measurement devices 100, registering and managing the health measurement devices 100, and updating device related information.

The user management module 280 stores and manages information about a user who uses the health measurement device 100.

The data management module 290 manages the user health data obtained through the health measurement device 100 and the measurement module 260, and the feedback data received from the health management service server 300.

FIG. 6 is an internal block diagram of the health management service server in the system for providing health management service of FIG. 3.

Referring to FIG. 6, the health management service server 300 includes a LAN 310, a protocol module 320, a user management module 330, a data management module 340, a data analysis module 350, an additional information providing module 360, and a control module 370.

The LAN 310 connects the health management service server 300 to the health management station 200 through the Internet. Here, although the invention shows a LAN is used as an interface for connecting the health management station 200 with the health management service server 300, a WLAN or wireless application protocol (WAP) conforming to the 802.11 standard may also be used as the interface.

The protocol module 320 converts the user ID information and the user health data obtained through the measurement module 340 into a form that can be available on the health management station 200 or the data management module 350.

The user management module 330 stores and manages information about a user who uses the health measurement device 100 and the health management station 200.

The data management module 340 stores and manages the user health data received from the health management station 200, analyzed result and feedback data received from the data analysis module 350.

In addition, the data management module 340 guides information received from the health guide station 400.

The data analysis module 350 analyzes the user's health status based on the user health data and generates feedback data based on the analyzed result. Here, the data analysis module 350 generates initial user feedback data based on the user demographic information and health data. For subsequent feedback data, the data analysis module 350 analyzes the previous feedback data and health data newly obtained through measurement and applies a weighted value to the analyzed result to generate new feedback data.

That is, when the result of analyzing current user health data newly obtained after providing feedback data (i.e. prescription) based on the previous user health data shows little or no improvement in the user's health status (e.g., weight, blood pressure, blood sugar, etc), the data analysis module 350 must provide a new prescription to the user. This is important, as if the same feedback data as the previous feedback data is again provided for re-prescription, instead of the new feedback data obtained by assigning the weighted value, it is impossible to improve the user's health status.

Thus, the data analysis module 350 calculates a predetermined weighted value based on the previous feedback data and the current user health data and applies the weighted value for re-prescription. Here, the weighted value may vary depending on the number of re-prescriptions.

For example, a method for applying a weighted value to a meal menu for a user who desires to adjust his/her weight will now be described using the following Equations (1) and (2):

$$\begin{matrix}{y}_{a} = f ( {x}_{1 a} , {x}_{2 a} ) & ( 1 ) \\ Standard dietary prescription \times \frac{{x}_{2 i}}{{x}_{2 c}} & ( 2 )\end{matrix}$$

The amount of user's caloric intake is calculated using the Equation (1) and the amount of user's caloric intake obtained by assigning a weighted value is defined by Equation (2). In Equation (1), yₐ, X₁ₐ, and X₂ₐ, denote user's weight, the amount of calories consumed by the user, and the amount of the user's caloric intake, respectively. In Equation (2), X₂ᵢ, and X_{2c} denote the amount of caloric intake entered by the user and the amount of caloric intake calculated using the Equation (1), respectively.

That is, the amount of user's caloric intake can be obtained by substituting the user's weight and the amount of calories consumed (or amount of exercise) that can be measured more precisely than the quantity of food items consumed by the user into the Equation (1).

For example, when user A may think he/she takes in 100 calories but he/she actually takes in 120 calories, he/she may complain that his/her weight failed to reach the target weight loss in spite of compliance with given prescription.

However, the failure occurs due to the kind of ingredients in food, the kind of food made from ingredients, the kind of vessel containing the food, the type of food container, or individual difference in absorption of food rather than a user or system error.

Thus, the data analysis module 350 applies a weighted value upon prescribing a dietary menu for the user, thus helping the user to achieve target weight loss.

That is, substituting the weight and the amount of calories consumed (or amount of exercise) into yₐ and X₁ₐ in the Equation (1) gives the amount of the user's caloric intake X2a.

Then, the amount of the user's caloric intake X₂ₐ (e.g., 120) is compared with the amount of caloric intake entered by the user X₂ᵢ(e.g., 100). This comparison shows that the user misunderstood the current amount of his/her caloric intake.

The data analysis module 350 provides the amount of calories consumed multiplied by the weighted value (100/120=0.8) calculated through the Equation (2) to the user for re-prescription, thus allowing the user to easily reach a target weight set by him/her.

The weighted value can also be applied to re-prescription for other health measurables, such as blood sugar or blood pressure. Furthermore, the weighted value can also be applied to an exercise prescription.

The additional information providing module 360 provides necessary additional information (e.g., information about an off-line service provider that supplies related product information and/or services) based on the user health data and feedback data.

The control module 370 checks information about a predetermined user and data information and transmits the same information to the data analysis module 350, thus allowing the data analysis module 350 to assign a weighted value in generating subsequent feedback data for the user.

The control module 370 also controls the operations of components of the health management service server 300.

Meanwhile, the data analysis module 350 and the additional information providing module 360 may be implemented as separate service provider servers.

FIG. 7 is an internal block diagram of the health guide station in the system for providing health management service of FIG. 3.

Referring to FIG. 7, the health guide station 400 includes a device connection module 410, a user authentication module 420, a protocol module 430, a measurement module 440, a data management module 450, an input module 460, and a display module 470.

The device connection module 410 receives user health data from the health management service server 300 or transmits guide information input through the input module 460 to the health management service server 300. Here, the guide information can be understood as inquires written by a health planner, the details of consultations with a user, and user health data obtained through the measurement module 440. The device connection module 410 may be understood as a wireless connection module such as a Code Division Multiple Access (CDMA) or WLAN module.

The user authentication module 420 reads user ID information from the authenticating device 500 to authenticate the user. That is, the user authentication module 120 reads user information and user authentication key from a RFID tag in the authenticating device 500 in order to authenticate the user.

The protocol module 430 transmits health data obtained by measuring the user's heath status, guide information, and user information. That is, the protocol module 430 converts the user ID information and the user health data obtained through the data measurement module 450 into a form that can be available on the health management station 300, and transmits the resulting information to the device connection module 410.

The measurement module 440 measures a user's health status to generate health data that is then stored in the data management module 450 and the health management service server 300.

The data management module 450 manages the user health data received from the measurement module 440 and the health management service server 300.

The input module 460 receives predetermined information such as the details of inquires, consultations, and prescription from the health planner.

The display module 470 displays the user health data and feedback data received from the health management service server 300 and the user health data obtained by the measurement module 440.

FIG. 8 is an internal block diagram of the authenticating device in the system for providing health management service of FIG. 3.

Referring to FIG. 8, the authenticating device 500 includes a data transceiving module 510, a storage module 520, and a control module 530.

The data transceiving module 510 transmits and receives user demographic information and health data obtained from the health measurement device 100, the health management station 200, and the health guide station 400. The input module 230 receives predetermined information such as demographic information (e.g., age, gender, etc.) and health status information (e.g., height, weight, blood pressure, etc.) from a user.

The storage module 520 stores various information about a user and is divided into: (1) an encryption key information region storing encryption key information for user authentication; (2) a personal information region storing user's personal information; (3) a measurement result information region storing health data obtained through measurement and (4) a prescription information region storing the details of a prescription made based on the user's health status.

The control module 530 allows the data transceiving module 510 to transmit user information when the user uses the health measurement device 100 and the health guide station 400 and to receive user information and guide information obtained through the health measurement device 100 and the health guide station 400 while allowing the storage module 520 to store the received information.

FIG. 9 is a diagram showing a system for providing a health management service consisting of a health management service server and external service servers according to a second exemplary embodiment of the invention.

As shown in FIG. 9, the health management service sever 300 interworks with a plurality of external service servers 710 and 720 (collectively denoted by 700). Here, the external service server 700 may be understood as being an insurance company, a clinic center, and/or a fitness health club.

The health management service server 300 analyzes user health data received from the health measurement device and the health management station 200 and generates the analyzed result and feedback data based on the analyzed result while transmitting user information, the health data, and the feedback data to the external service server 700. The external service server 700 searches for information about services suitable for the user based on the received user information, health data, and feedback data and transmits the retrieved service information to the user.

Here, the external service server 700 provides the retrieved service suitable for the user by linking a service provider providing the service to the health management service server 300, transmitting the service using Short Message Service (SMS), or performing on/off line consultations with the user. The external service server 700 also provides discount benefits for the user when he/she uses an external service provider supported by the external service provider. Thus, the external service server 700 allows the user to easily purchase necessary products and obtain health information suitable for his/her health status and prescription as well as discount benefits upon purchase of products from an external service provider

For example, when the external service server 700 is an insurance company, the insurance company receives user information, health data, and feedback data from the health management service server 300. The insurance company searches for an insurance product suitable for user's health status based on the received user information, health data, and feedback data, and transmits the found insurance product to a user's portable terminal using SMS. The user that receives information about the insurance product suited for his/her health status applies for the received insurance product upon purchase. Thus, this enables the user to choose the most suitable insurance product while receiving discount benefits upon purchase.

When the external service server 700 is a school or company, the health management service server 300 provides health management information and prescriptions for students or office workers. The external service server 700 can utilize the health status information about students or office workers provided by the health management service server 300 as material for student cumulative record or worker's personal record.

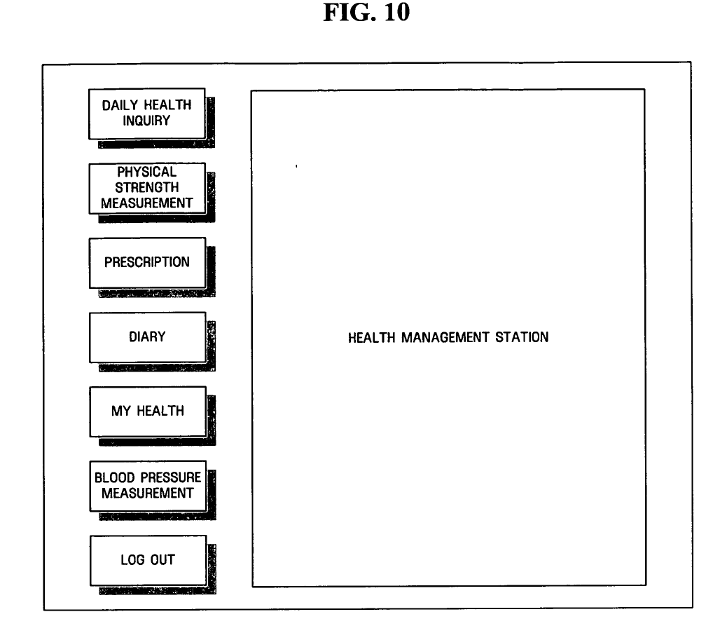

Referring to FIG. 10, the health management station 200 displays a health management screen, including health information and multiple menu items for health management, to a user. For example, “Daily Health Inquiry” contains the details of an inquiry about the current user's health status. “Physical Strength Measurement” specifies information about general physical strength measurements (e.g., muscular endurance, cardio-pulmonary endurance, flexibility, etc.). “Prescription” describes the details of a prescription (i.e., feedback data) provided for the user and “Diary” contains the amount of user's caloric intake (i.e., quality of food consumed) and the amount of exercise. “My Health” contains the user's health record and “Blood Pressure Measurement” describes the user's blood pressure measured. “Log In/Out” indicates whether the user is logged on or out. Each item is implemented in the form of a touch pad and changed to a screen linked to an appropriate item upon user's selection. Other kinds of items may also be displayed on the health management screen.

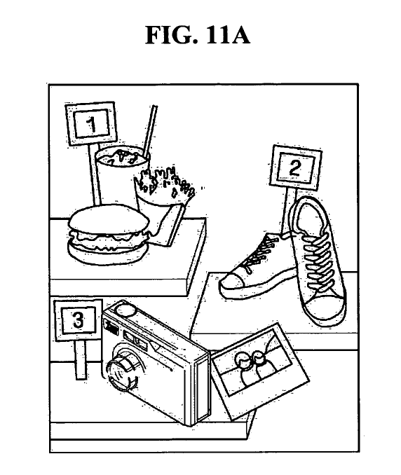

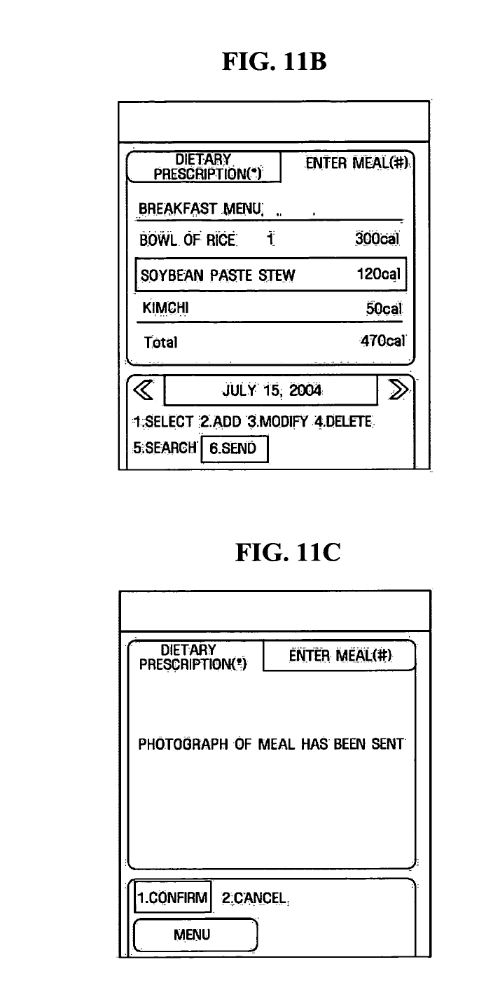

FIGS. 11A-11C illustrate a process of providing information about food items in a user's meal using a mobile device according to an exemplary embodiment of the invention.

The user takes a picture of food items that he/she has eaten using a mobile device (e.g., a mobile phone equipped with a camera) and transmits the picture to the health management service server 300. To compensate for the drawbacks of the mobile device equipped with an interface in which information input is inconvenient, the invention allows the user to transmit only an image of food instead of entering each food item that the user has consumed and to view the image from the Diary item on the screen of the health management station 200. The invention also enables the user to cumulatively manage information concerning the amount of the user's caloric intake and the amount of calories burned by the user, thereby enabling consistent monitoring of compliance with dietary prescription. Further, the invention allows images of various kinds of food to be displayed through a mobile phone, thereby providing entertainment to the user.

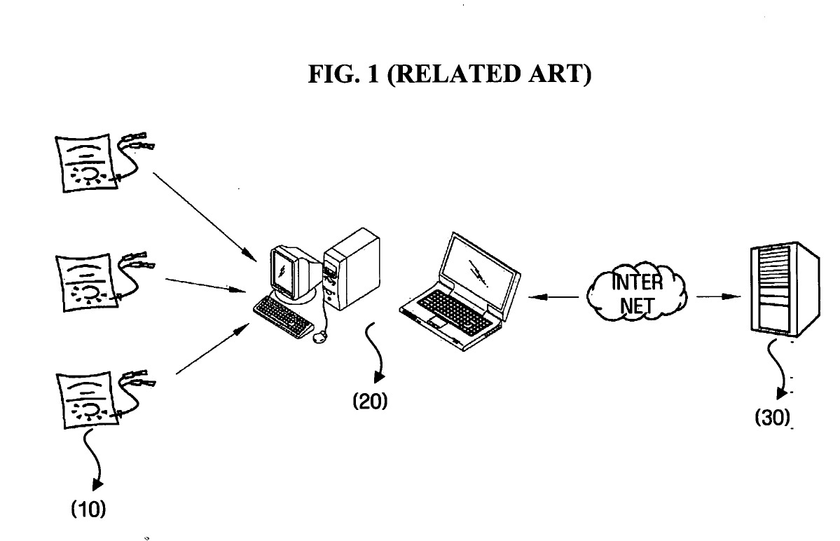

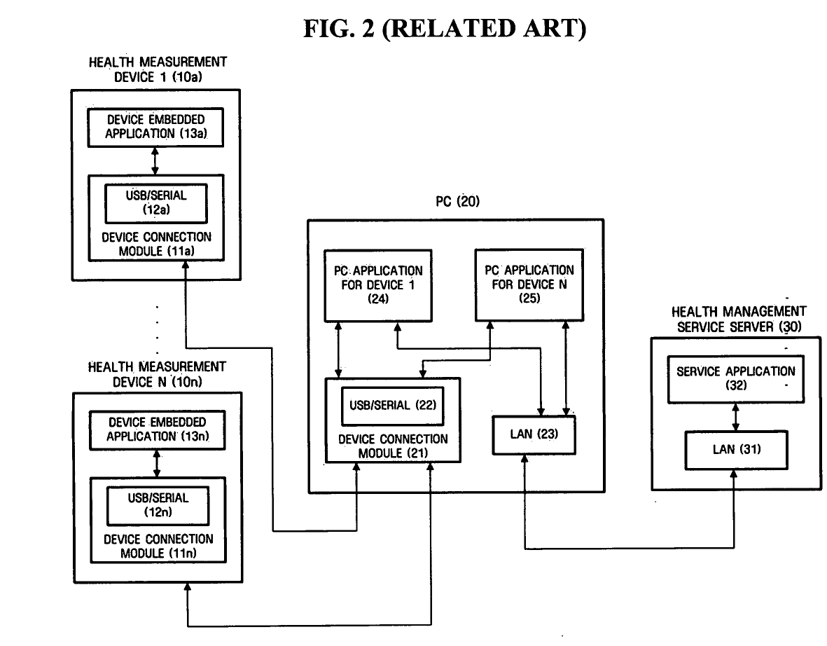

For example, as shown in FIG. 1I A, the user selects a displayed food image from a mobile phone's screen to enter information about food items that he/she has eaten. When the mobile phone's screen changes to a screen shown in FIG. 11B, the user enters information about food items that he/she has eaten and then selects “Transmit.” The screen then changes to a screen shown in FIG. 11C. In this way, the user can easily transmit the information about food items consumed to the health management service server 300 while the health management service server 300 uses the received information to generate the user's feedback data.

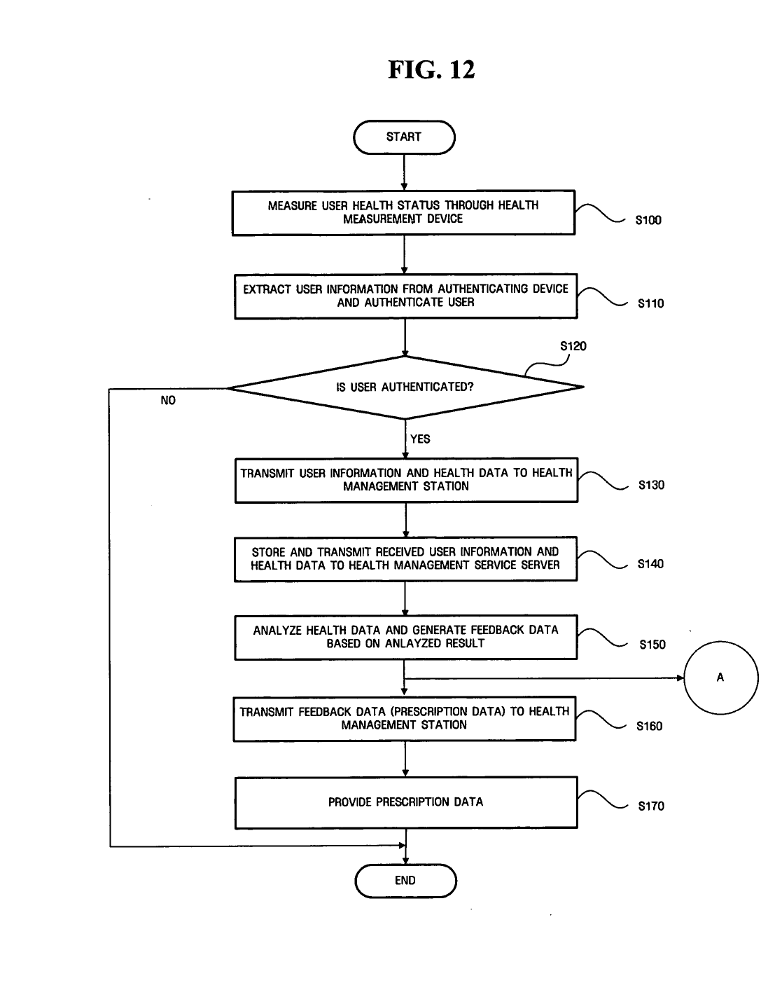

FIG. 12 is a flowchart illustrating a method for providing health management service according to an exemplary embodiment of the invention. Here, since a user has subscribed to a health management service through the health management station 200, user information and device information of the health measurement device 100 are stored in the health management station 200 and the health management service server 300.

In operation S100, when the user operates the health measurement device 100, the measurement module 140 of the health measurement device 100 measures the user's health status. In operation S110, the user authentication module 120 recognizes the authenticating device 500 (i.e. an RFID card) held by the user, reads user ID information, and uses the user ID information to authenticate the user.

When the user is authenticated in operation S120, the protocol module 130 converts the user information extracted by the user authentication module 120 and health data obtained through the measurement module 140 into a form that can be available on the user management module 150 and transmits the converted user information and health data to the user management module 150.

The user management module 150 then stores the health data as information related to the appropriate user and transmits the user information and the health data to the device connection module 110. In this case, the user information and the health data are converted by the protocol module 130 into a form that can be available on the health management station 200 before being transmitted to the device connection module 110.

Then, in operation S130, the device connection module 110 transmits the user information and health data to the health management station 200 through a Bluetooth or WLAN connection.

Then, the device connection module 210 of the health management station 200 receives the user information and the health data from the health measurement device 100. The protocol module 250 converts the received user information and health data into a form that can be available on the user management module 280 and the data management module 290 and transmits the converted user information and health data to the user management module 280 and the data management module 290.

The user management module 280 and the data management module 290 store the received user information and health data and then transmits the same to the LAN 220.

In operation S140, the user information and health data are transmitted to the health management service server 300 through the LAN 220.

Then, in operation S150, the data analysis module 350 analyzes the transmitted health data and generates feedback data based on the analyzed result.

In operation S160, the health management service server 300 transmits the generated feedback data (i.e., prescription data) to the health management station 200 via the LAN 310. In this case, the feedback data (i.e., prescription data) is converted by the protocol module 320 into a form that can be available on the health management station 200 before being transmitted to the LAN 310.

In operation S170, the feedback data (i.e., prescription data) received through the LAN 220 of the health management station 200 is provided to the user through the display module 240. Here, the feedback data (i.e., prescription data) may be provided through a predetermined health measurement device 100 or the mobile health measurement device 600 as well.

This method enables the user to check his/her health status through the feedback data (i.e., prescription data) and receive health management service through the system for providing a health management service.

Furthermore, the health management service server 300 provides user's health information and prescription details to off-line service providers providing health related products and services so that the off-line service providers can propose services suited for the user's health status, thereby enabling the user to easily comply with a prescription to change health management/dietary/exercise behavior.

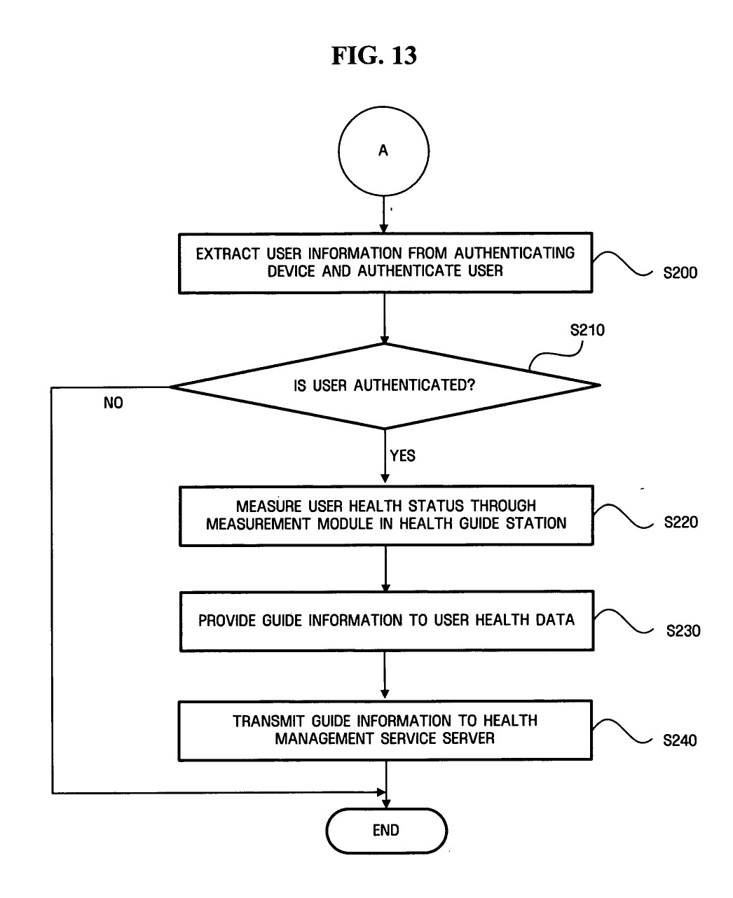

FIG. 13 is a flowchart illustrating the process of providing health management service through a health guide station in the method for providing health management service according to an exemplary embodiment of the invention. It is assumed that the user health data received from the health measurement device 100 or health management station 200 are stored and managed in the health management service server 300.

In operation S200, when a health planner takes the authenticating device 500 (i.e., an RFID card) held by a user to the health guide station 400.

Then, the user authentication module 420 of the health guide station 400 recognizes the authenticating device 500, reads user ID information, and uses the recognized user ID information to authenticate the user.

When the user is authenticated in operation S210, the display module 470 displays that the user is authenticated on a screen.

Then, in operation S220, the health planner measures the user's health status through the measurement module 440 of the health guide station 400. The measurement module 440 provides user health data to the screen through the display module 470.

In operation S230, the health planner provides exercise recommendations and guide information about dietary habit to the user based on the health data.

In operation S240, the health planner enters guide information about the user through the health guide station 400 and transmits the entered guide information to the health management service server 300 through the device connection module 410. Here, the health management service server 300 stores the guide information received from the health guide station 400 in the data management module 340.

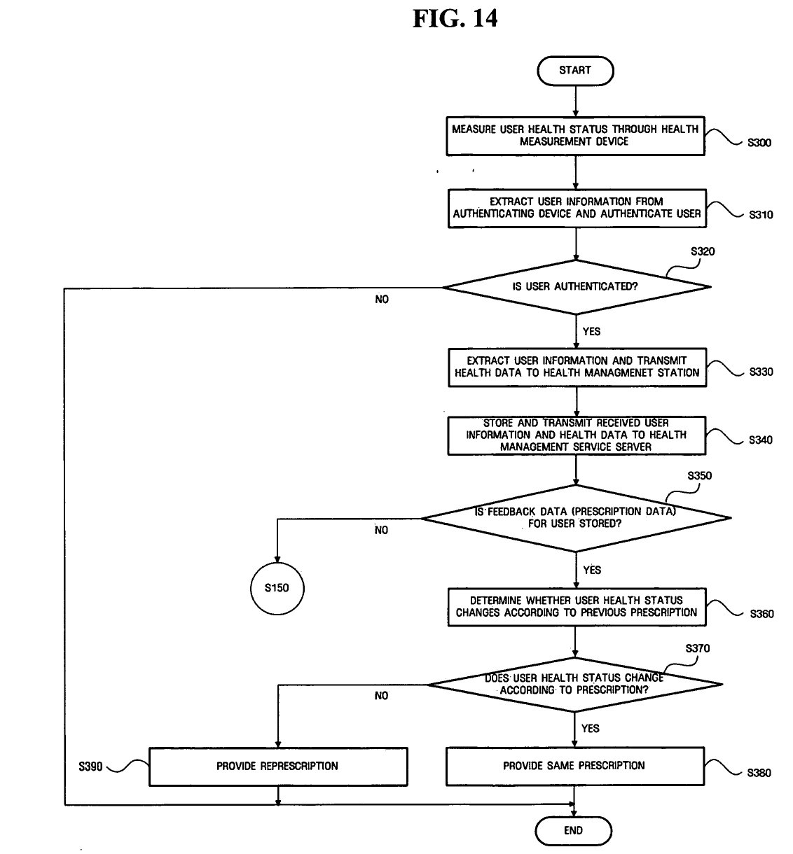

FIG. 14 is a flowchart illustrating the process of providing a re-prescription service to the user in the method for providing health management service according to an exemplary embodiment of the invention.

In operation S300, when the user operates the health measurement device 100, the measurement module 140 of the health measurement device 100 measures user's health status.

In operation S310, the user authentication module 120 recognizes the authenticating device 500 (i.e. a RFID card) held by the user, reads user ID information, and uses the recognized user ID information to authenticate the user.

When the user is authenticated in operation S320, the protocol module 130 converts the user information extracted by the user authentication module 120 and health data obtained through the measurement module 140 into a form that can be available on the user management module 150 and transmits the converted user information and health data to the user management module 150.

The user management module 150 then stores the health data as information of the appropriate user and transmits the user information and the health data to the device connection module 110. In this case, the user information and the health data are converted by the protocol module 130 into a form that can be available on the health management station 200 before being transmitted to the device connection module 110.

Then, in operation S330, the device connection module 110 transmits the user information and the health data to the health management station 200. Here, the device connection device 110 transmits the user information and the health data through Bluetooth or WLAN.

Then, the device connection module 210 of the health management station 200 receives the user information and the health data from the health measurement device 100. The protocol module 250 converts the received user information and health data into a form that can be available on the user management module 280 and the data management module 290 and transmits the converted user information and health data to the user management module 280 and the data management module 290.

Then, in operation S340, the user management module 280 and the data management module 290 store the received user information and health data, which are then also transmitted to the health management service server 300 through the LAN 220. Data analysis module 350 then analyzes the transmitted health data and generates feedback data based on the analyzed result.

Then, in operation S350, when the user management module 330 and the data management module 340 store the user information and health data, the control module 370 checks whether feedback data for the user is stored in the data management module 340.

In operation S360, when the feedback data for the user exists, the control module 370 transmits the user feedback data and health data to the data analysis module 350 and the data analysis module 350 compares the health data with the feedback data for analysis and determines whether the user's health status has changed according to the previous prescription (feedback data).

When the user's health status has changed according to the previous prescription in operation S370, the data analysis module 350 analyzes the received health data to generate feedback data (that is the same as the previous prescription data) based on the analyzed result in operation S380.

Conversely, when the user's health status has not changed according to the previous prescription in operation S370, the data analysis module 350 analyzes the previous feedback data and the current health data and assigns a weighted value calculated based on the analyzed result to generate new feedback data (i.e., re-prescription) in operation S390. Because the process of generating feedback data to which a weighted value is assigned is as shown in FIG. 6, detailed explanation thereof will not be given.

On the other hand, when the feedback data for the user does not exist in operation S350, the operations S150 and S170 illustrated in FIG. 12 are performed.

Thus, the method for providing health management service can provide re-prescription to the user by monitoring the user's health status and assigning a weighted value calculated based on the result of monitoring instead of providing health management service through fragmentary health data, thereby allowing for heath management and prescription tailored to an individual's needs.

A process of providing a weight management service according to an exemplary embodiment of the invention will now be described. It is assumed in the embodiment that a 42-year old man A with obesity receives weight management service.

First, the man A applies for a weight management service, registers health information (e.g., height, weight, and body fat percentage) including personal information, and enters medical history, dietary habit, exercise habit, and basic physical strength through an on-line inquiry. The basic physical strength needs a separate examination before being entered.

Then, the man A receives data about ideal weight and body fat percentage from the health management service server 300 based on the entered data and sets and enters a target weight loss and a target weight loss period through the health management station 200. Here, the target weight loss of 6 to 12 kg is set for the target weight loss period of 12 weeks.

The man A receives the amount of calories to be reduced for a week calculated based on the target weight loss for 12 weeks from the health management service server 300 through the health management station 200. Here, the amount of calories to be reduced for a week is obtained by converting the target weight loss for a week into calories (1 kg=7,700 kcal) in the range of 3,500 to 7,000 calories.

Further, the man A receives exercise prescription based on the basic physical strength information (e.g., cardiopulmonary endurance) obtained from the health management service server 300 through separate examination and the previously entered health information. Here, the exercise prescription is made by comprehensively considering the type of exercise and intensity and includes daily/weekly duration, frequency (times per day/week), and intensity.

Meanwhile, the health management service server 300 monitors man A's compliance with the exercise prescription through an amount of exercise by entering information about an amount of daily exercise.

Then, the man A receives the amount of caloric intake to be reduced for 1 week from the health management service server 300. Here, the amount of caloric intake to be reduced for 1 week is obtained by subtracting the amount of calories needed to meet the exercise prescription for the week from the overall amount of calories to be reduced for the week.

Further, the daily amount of calories needed to meet dietary prescription is calculated based on the amount of caloric intake to be reduced for 1 week by subtracting the daily amount of caloric intake to be reduced from the daily amount of energy required. The man A is provided with dietary prescription based on the daily amount of calories needed to meet the dietary prescription.

Here, the dietary prescription may contain several choices by comprehensively considering individual preferences and past dietary habits. The user may select a weekly dietary menu.

The man A then may enter the amount of the food items that he has eaten for a day so that the health management service server 300 performs monitoring based on the entered amount.

The health management service server 300 also performs monitoring on the exercise prescription and the dietary prescription and provides new dietary prescription obtained by assigning a weighted value to the man A when he fails to comply with prescription requirements, thus allowing for more efficient weight management.

Thus, the man A can be provided with weight management service suitable for his health status and a re-prescription made by the health management service server 300 through monitoring, thereby achieving more efficient weight management.

Meanwhile, the health management service can be provided to an individual as well as an entity. A process of providing health management service to a predetermined entity will now be described.

For example, a service may be arranged to provide health information about the members in a certain entity, and a personal management system of the entity may be connected to the health management service server 300.

Then, common weight/body fat scale, blood pressure measurement device, and blood sugar measurement devices may be installed at a commonly accessible location at the entity.

Then, each of the members in the entity may measure his/her physical/biological information and transmit the measured information to the health management service server 300.

The health management service server 300 may then analyze health data for an individual member in the entity and transmit a service that is appropriate for the member's health status to a personal management system in the entity.

Thus, the member in the entity is provided with his/her health status and feedback data and complies with exercise and dietary guidelines according to the feedback data. Here, the entity may also use information about the meals and health management of its members based on the feedback result as a material for personal management.

Meanwhile, the health management service server 300 may provide information about its affiliated off-line service providers (e.g., a clinic center, a fitness health club, an insurance company, etc.) to the entity. When using the off-line service providers, the entity can receive discount benefits.

When a member is found to have health problem, the health management service server 300 also automatically makes a reservation with its affiliated hospital for examination, and notifies reservation details to the entity or the member.

As described above, the invention provides re-prescription to a user by monitoring user health data and feedback data and assigning a weighted value to the result of monitoring, thereby providing health management service and prescription suitable for the user's health status while allowing for accurate health management and prescription through consistent monitoring instead of based on fragmentary health data.

The invention also allows a health planner to provide one-to-one guidance on/off line based on user health data.

Meanwhile, a health management service server interworks with an external service provider so that the external service provider can provide products and information suitable for the user based on user information and health information (e.g., health data, feedback data, etc), thus allowing the user to easily purchase necessary products and obtain health information appropriate for his/her health status and prescription as well as discount benefits upon purchase of products from the external service provider.

While the invention has been particularly shown and described with reference to exemplary embodiments thereof, the invention is not limited to these embodiments. It will be understood by those of ordinary skill in the art that various changes in form and details may be made therein without departing from the spirit and scope of the invention as defined by the following claims.

What is claimed is:

1. A health measurement device comprising:
a measurement module which measures a health status of a user;
a user authentication module which authenticates the user;
a user management module which manages information about the user and health data of the user obtained through the measuring of the health status of the user; and
a protocol module which transmits the health data and the information.

2. A health management station comprising:
a user management module which manages information concerning a user who uses a health measurement device;
a data management module which manages health data of the user obtained through measurement of a health status of the user and feedback data received from a health management service server; and
a protocol module which transmits the health data and the feedback data.

3. The health management station of claim 2, further comprising a measurement module which measures the health status of the user.

4. The health management station of claim 2, further comprising an input module which receives further information about the health status of the user.

5. A health management service server comprising:
a user management module which manages information about a user received from a health management station;
a data analysis module which analyzes the information and health data of the user received from the health management station, and generates feedback data based on a result of analyzing the information and the health data;
a data management module which manages the health data and the feedback data; and
a protocol module which transmits the health data and the feedback data.

6. The health management service server of claim 5, wherein the data analysis module generates the feedback data by assigning a weighted value according to compliance with previously provided feedback data.

7. A health guide station comprising:
a device connection module which transmits guide information received from a health planner;
a user authentication module which authenticates a user;
a data management module which manages the guide information, and health data of the user and feedback data received from a health management service server; and
a protocol module which transmits the guide information and the health data.

8. The health guide station of claim 7, further comprising a measurement module to measure a health status of the user.

9. The health guide station of claim 7, wherein the health data and the feedback data are received from the health management service server to generate the guide information.

10. A system for providing a health management service, the system comprising:
a health measurement device which measures a health status of a user and transmits health data of the user obtained through the measurement;
a health management station which receives the health data from the health measurement device, transmits the health data, and manages feedback data; and
a health management service server which receives the health data from the health management station, and generates feedback data based on the health data, and transmits the feedback data to the health management station.

11. The system of claim 10, further comprising a health guide station which transmits guide information received from a health planner to the health management service server.

12. The system of claim 10, wherein the health measurement device comprises:
a measurement module which measures the health status;
a user authentication module which authenticates the user;
a user management module which manages the health data and information about the user; and
a protocol module which transmits the health data and the information.

13. The system of claim 10, wherein the health management station comprises:
a user management module which manages information concerning the user;
a data management module which manages the health data and the feedback data received from the health management service server; and
a protocol module which transmits the health data and the feedback data.

14. The system of claim 13, further comprising a measurement module which measures the health status.

15. The system of claim 10, wherein the health management service server comprises:
a user management module which manages information about the user received from the health management station;
a data analysis module which analyzes the information and the health data received from the health management station, and generates the feedback data based on a result of analyzing the information and the health data;
a data management module which manages the health data and the feedback data; and
a protocol module which transmits the health data and the feedback data.

16. The system of claim 15, wherein the data analysis module generates the feedback data by assigning a weighted value according to compliance of the user with previously provided feedback data.

17. The system of claim 11, wherein the health guide station comprises:
a device connection module which transmits the guide information received from the health planner;
a user authentication module which authenticates the user;
a data management module which manages the guide information, and the health data and the feedback data received from the health management service server; and
a protocol module which transmits the guide information and the health data.

18. The system of claim 17, wherein the health guide station further comprises a measurement module to measure the health status of the user.

19. The system of claim 11, further comprising an authenticating device which stores information about the user and transmits the information to the health measurement device and the health guide station.

20. A system for providing a health management service, the system comprising:
a health management service server which analyzes health data of a user and manages a result of the analysis and feedback data generated based on the result of the analysis; and
an external service server which receives information about the user, the health data, and the feedback data from the health management service server and provides service appropriate for the user based on the information, the health data, and the feedback data.

21. The system of claim 20, wherein the external service server provides the service appropriate for the user by linking a service provider providing the service to the health management service server, transmitting an identification of the service to a terminal of the user using Short Message Service (SMS), or performing on/off line consultations with the user.

22. A method for providing a health management service, the method comprising:
measuring a health status of a user through a health measurement device;
authenticating the user;
transmitting health data obtained through the measurement of the health status and information about the user to a health management station;
storing the health data and the information;
transmitting the health data and the information to a health management service server;
generating feedback data at the health management service server; and
transmitting the feedback data to the health management station.

23. The method of claim 22, further comprising:
generating guide information based on the health data; and
transmitting the guide information to the health management service server.

24. The method of claim 23, wherein the guide information is generated through further measurement of the health status of the user.

25. The method of claim 23, further comprising requesting the health management service server to transmit health data and feedback data associated with the user.

26. The method of claim 22, further comprising:
determining whether feedback data for the user exists;
checking compliance of the user with the feedback data when the feedback data exists; and
generating new feedback data based on the feedback data and the health data when the user fails to comply with the feedback data.

27. The method of claim 26, wherein the new feedback data is generated by assigning a weighted value.

28. A method for providing a health management service, the method comprising:
receiving information about a user, health data of the user, and feedback data from a health management service server;
searching for information concerning a service suitable for the user based on the information, the health data, and the feedback data, which are received; and
providing the service information to the user.

29. The method of claim 28, wherein the service information is provided to the user by: linking a service provider providing the service to the health management service server; transmitting an identification of the service to a terminal of the user using Short Message Service (SMS); or performing on/off line consultations with the user.
